(19) United States
(12) Patent Application Publication
(10) Pub. No.: US 2025/0177652 A1
(43) Pub. Date: Jun. 5, 2025

(54) DRIVE MECHANISM FOR A DRUG DELIVERY DEVICE

(71) Applicant: Sanofi, PARIS (FR)

(72) Inventors: Alexander Allerdings, Frankfurt am Main (DE); Michael Schabbach, Frankfurt am Main (DE); Meinolf Werner, Worms (DE)

(21) Appl. No.: 18/843,975

(22) PCT Filed: Mar. 9, 2023
(86) PCT No.: PCT/EP2023/055984

§ 371 Date: Sep. 4, 2024

(30) Foreign Application Priority Data
Mar. 11, 2022 (EP) 22315056.6

(51) Int. Cl. A61M 5/24; A61M 5/315

(52) U.S. Cl. CPC A61M 5/2422; A61M 5/31511; A61M 5/31578; A61M 2005/2485; A61M 2005/31588; A61M 2202/0007; A61M 2202/04; A61M 2205/10

(57) ABSTRACT

The present disclosure relates to a drive mechanism (11) for moving a stopper (24) of a medicament container (22), wherein the stopper (24) is movably disposed inside a longitudinally extending barrel (23) of the medicament container (22) and is movable in a longitudinal direction (z) relative to the barrel (23), the drive mechanism (11) comprising: —a receptacle (30) sized to receive the medicament container (22), —a first electromagnet (41) comprising an electromagnetic coil (42) and a core (44) extending through the electromagnetic coil (42), wherein a radial center (43) of the electromagnetic coil (42) is located outside the receptacle (30), —a magnetic coupling member (80) mechanically engagable with the stopper (24) and configured for insertion into the barrel (23), —wherein the first electromagnet (41) is operable to generate a magnetic field causing the magnetic coupling member (80) to move in the longitudinal direction (z).

### FIELD

The present disclosure relates to the field of drug delivery devices, in particular for delivery of liquid medicaments, e.g. by way of infusion or injection. In one aspect the present disclosure relates to a drive mechanism for moving a stopper of a cartridge filled with a liquid medicament.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump.

With drug delivery device or injection devices a liquid medicament is typically provided in a medicament container such as a cartridge, a syringe or carpule. Such medicament containers are typically closed or sealed towards a proximal direction by way of a stopper movably disposed in a barrel of such medicament containers. The liquid medicament can be expelled or dispensed from the medicament container by displacing, e.g. urging the stopper in distal direction relative to the barrel of the container. The distal outlet of the medicament containers may be in fluid connection with an injection needle or infusion line.

For introducing or transferring a driving force into or onto the stopper there exist mechanically implemented drive mechanisms, e.g. comprising an elongated piston rod in longitudinal abutment with a proximal end face of the stopper. Drive mechanisms known so far are typically operable to advance the piston rod in distal direction so as to exert a dispensing pressure onto the stopper of the medicament container.

With increasing digitalization and with the availability of electrically implemented drive mechanisms, i.e. drive mechanisms, wherein a force for moving the stopper in a distal direction is provided or generated by an electric or electromechanical drive, there is an increasing demand for new solutions and approaches of how to provide a mechanical coupling between the driver or drive member of the drive mechanism with the stopper of the medicament container, e.g. with the stopper of a cartridge. Moreover, it is also desirable to miniaturize such drive mechanisms and drug delivery devices. The drive mechanism, in particular the engagement or interface between a component of a drive mechanism and the stopper of the medicament container to be moved by the drive mechanism should be rather failure safe, robust and efficient in terms of a longitudinal force transfer.

### SUMMARY

In one aspect the present disclosure relates to a drive mechanism for moving a stopper of a medicament container, e.g. a stopper of a medicament cartridge. The stopper is movably disposed or movably arranged inside a longitudinally extending barrel of the medicament container. The stopper is movable in a longitudinal direction relative to the barrel, e.g. for expelling a dose of a liquid substance from the barrel. The drive mechanism comprises a receptacle sized to receive the medicament container. The receptacle may be configured to hold the medicament container and/or to fix the medicament container inside the receptacle.

The drive mechanism further comprises a first electromagnet. The first electromagnet comprises an electromagnetic coil and a core extending through the electromagnetic coil. A radial center of the electromagnetic coil is located outside the receptacle. The drive mechanism further comprises a magnetic coupling member mechanically engageable with the stopper and configured for insertion into the barrel. The first electromagnet is further operable to generate a magnetic field causing the magnetic coupling member to move in the longitudinal direction.

Typically, the entire electromagnet is located outside the receptacle or adjoins the receptacle in radial direction. Here, the radial direction extends perpendicular to the longitudinal direction. Generally and in the present context a radial direction may refer to a cylindrical geometry of the barrel of the medicament container and hence to a cylindrical geometry of the receptacle. However, the present disclosure is not restricted to receptacles and/or medicament containers of tubular shape. In this regard, a radial direction generally has to be regarded as a direction pointing to a center of a cross-section of the receptacle, which cross-section extends perpendicular to the longitudinal direction, along which the stopper is movable relative to the barrel of the cartridge. Respective considerations apply to the expressions of a tangential or circumferential direction. These extend perpendicular to the longitudinal direction and to the radial direction.

Generation of the magnetic field is caused by applying an electric current to the first electromagnet. The electromagnetic coil, e.g. comprising a solenoid, is operable to generate a magnetic field, which is amplified by the core extending through the hollow interior of the electromagnetic coil. This way and by using an electromagnet comprising an electromagnetic coil and a core, the magnetic field generated by the electromagnetic coil can be amplified. In effect, a larger magnetic force can be applied to the magnetic coupling member. Moreover, and since the radial center of the electromagnetic coil is located outside the receptacle the entire electromagnet may be arranged outside the receptacle. This provides a rather compact and robust design of the drive mechanism.

By making use of a core extending through the electromagnetic coil, the number of windings of a coil as well as a magnitude of an electric current to be applied to the coil can be substantially reduced without any detrimental effect on the strength of the magnetic field interacting with the magnetic coupling member. Also, and in this way, constructional space for the drive mechanism can be reduced, thus allowing to further miniaturize the drive mechanism and/or a drug delivery device equipped with such a drive mechanism.

By arranging at least the center of the electromagnetic coil outside the receptacle the first electromagnet does not necessarily have to contribute to the receptacle. Hence, the first electromagnet may be located radially next to or radially adjacent and outside the receptacle. In this way, the first electromagnet does not necessarily have to be individually designed or shaped to match with a receptacle of a given size. This way, the first electromagnet may be universally usable for a large variety of differently shaped receptacles. Here, a receptacle may conform or may be adapted and/or configured in accordance to the geometry or shape of the medicament container intended for insertion into the receptacle.

The core is typically implemented as an iron core. It may comprise a ferromagnetic or ferrimagnetic material. It may be monolithically shaped. The core may comprise a sofr.iron material. Hence, the iron material of the core has a low carbon content and is easily magnetized and demagnetized with a small hysteresis loss.

According to a further example the radial center of the electromagnetic coil is located at a radial offset from a radial center of the receptacle. Hence, as seen in a radial direction the electromagnetic coil may be located entirely outside the receptacle. Furthermore, a radial distance between the radial center of the receptacle and the radial center of the electromagnetic coil may be larger than the sum of the radius of the electromagnetic coil and the radius of the receptacle.

With receptacles of a non-circular cross-section, this may apply accordingly with regard to the distance from a sidewall of the receptacle towards a transverse or radial center of the receptacle.

This way, the electromagnetic coil and optionally the entire electromagnet are located outside the receptacle. This offers and enables a rather easy assembly of the first electromagnet relative to the receptacle. Moreover, by having the electromagnetic coil arranged at a radial offset from the radial center of the receptacle and/or at a radial offset from an outside of the receptacle the electromagnetic coil is e.g. accessible from outside, thus offering an easy way of providing electrical contact to the electromagnetic coil.

Typically and according to a further example the electromagnetic coil is arranged parallel to the longitudinal direction of the receptacle or barrel of the medicament container. A center line or line of symmetry extending longitudinally through the coil and thus forming a symmetry axis for the coil is typically arranged parallel to the longitudinal direction of the receptacle.

With a further example the core comprises a first leg, a base section and a second leg. The base section extends through the electromagnetic coil. The first leg and the second leg extend in radial direction towards the receptacle. Typically, the first leg and the second leg are mutually interconnected by the base section. The base section comprises a longitudinal extent that is equivalent or only slightly longer than the longitudinal extend of the electromagnetic coil. The first leg and the second leg may extend along a longitudinal end face of the coil. With regard to the cylindrical geometry of the electromagnetic coil the first leg and the second leg extend radially from the base section of the core.

The base section extends in longitudinal direction from the first leg to the second leg. Typically, the first leg and the second leg extend along the same radial direction from the base section. In this way, the first leg and the second leg may extend parallel to each other. The first leg and the second leg may comprise an equal extension in the radial direction as well as in a tangential or circumferential direction with regards to the geometry of the coil or of the receptacle.

With a further example the first leg and the second leg are separated in longitudinal direction and are mutually interconnected via the base section. Moreover, the first leg and the second leg as well as the base section may be unitarily formed. Hence, the or may comprise a single piece, such that the first leg, the base section and the second leg are integrally formed.

With some examples the core comprises a U-shaped profile. The first leg and the second leg comprise a rather elongated straight shape. The first leg and the second leg are arranged flush and parallel to each other. The first and the second leg each comprise a first longitudinal end, representing a free longitudinal end. The oppositely located longitudinal ends of both legs are connected with opposite longitudinal ends of the base section. By separating the first leg and the second leg in longitudinal direction there may be provided opposite or different magnetic poles along the longitudinal direction of the medicament container. Typically, the first electromagnet is located and arranged outside the receptacle such that the free ends of the first leg and the second leg of the core face towards the receptacle so as to provide a magnetic north pole and a magnetic south pole that are separated along the longitudinal direction.

With some examples the magnetic coupling member comprises a magnet. The magnet comprises opposite poles, namely a north pole and a south pole. The opposite poles are provided at oppositely located longitudinal ends of the magnetic coupling member. For instance, one of a south pole and a north pole faces towards a distal direction, hence towards an outlet of the cartridge or receptacle the other one of a the south pole and the north pole faces in the opposite direction, i.e. in proximal longitudinal direction.

The magnetic coupling member may comprise a rod magnet or a ring magnet. It may be in mechanical engagement with the stopper of the medicament container. It may be in abutment with a proximally facing end face of the stopper. It may be directly or indirectly connected or fixed to the stopper or it may be embedded or integrated into the stopper. In effect and by the mechanical engagement between the magnetic coupling member and the stopper or bung a magnetic force applied by the first electromagnet onto the magnetic coupling member can be unalterably transferred into a respective movement of the magnetic coupling member and hence into a respective movement of the stopper relative to the barrel.

According to a further example the core is located radially outside the receptacle. A free end of the first leg and a free end of the second leg is/are facing radially inwardly towards the receptacle. Here, the free ends of the first and second legs may directly adjoin the receptacle. With some examples, the first leg and the second they may form or contribute to a sidewall of the receptacle in which the medicament container can be accommodated. This way, the first leg and the second leg may even provide a mechanical guiding, a mechanical enclosure and/or a mechanical support for the medicament container. The first leg and the second leg may radially enclose the medicament container inside the receptacle.

According to a further example at least one of the free ends of the first leg and the second leg comprises a radially inwardly facing end face with a concave shape as seen in a tangential direction. This way, and when the receptacle is of tubular shape the radially inwardly facing end face of at least one of the free ends of the first leg and the second leg may precisely adapt or conform to the shape of the receptacle and/or to the shape of the outside surface of the tubular shaped barrel.

With some examples the tangential extent of the legs may be larger than a longitudinal extent of the legs. Also, more than 25°, more than 45° or even more than 60° of the outer circumference of the medicament container can be confined by the radially inwardly facing end face of the respective legs.

According to a further example the drive mechanism comprises a second electromagnet longitudinally offset from the first electromagnet. The first electromagnet and the second electromagnet form a first longitudinally extending array of electromagnets. The array of electromagnets may not be limited to only a first and a second electromagnet. The array or row of electromagnets may comprise even more electromagnets. The array of electromagnets may comprise a number of electromagnets that is defined by the longitudinal extend of the medicament container or of the receptacle divided by the longitudinal extend of a single electromagnet.

Typically, the second electromagnet is conceptually identical to the first electromagnet. Also, the second electromagnet comprises an electromagnetic coil and a core. Typically, the first electromagnet and the second electromagnet are substantially identical. This way and for creating an array of electromagnets a number of individual and identical electromagnets can be assembled in a longitudinal row. The longitudinally extending row or array of electromagnets typically extends parallel to the longitudinal direction of the barrel or longitudinal direction of the receptacle.

The first electromagnet and the second electromagnet may be arranged longitudinally adjacent to each other. The second electromagnet may longitudinally adjoin the first electromagnet. Here, the second leg of the first electromagnet may adjoin a first leg of the second electromagnet. The first leg of the first electromagnet may face away the second electromagnet. The second leg of the second electromagnet may face away the first electromagnet. Apart from a first and a second electromagnet the array of electromagnets may comprise numerous further electromagnets, e.g. three, four, five, six or even more electromagnets.

By making use of a row or array of numerous electromagnets the longitudinal range of the drive mechanism can be increased or extended. Typically, and by having numerous electromagnets along the longitudinal direction the magnetic coupling member can be moved from a proximal end towards and to a distal end of the barrel; and vice versa.

According to a further example the drive mechanism comprises a third electromagnet tangentially offset from the first electromagnet. Also, the third electromagnet may be implemented conceptually identical to the first electromagnet. It may be substantially identical to the first electromagnet. As seen from the center of the longitudinal receptacle the third electromagnet may be arranged at another circumferential portion of the receptacle. It may be hence tangentially offset from the first electromagnet. It may be located diametrically opposite to the first electromagnet and may adjoin the receptacle in radial direction. This way, the receptacle may be located radially between the first electromagnet and the third electromagnet.

By way of a first and a third electromagnet located on opposite sides of the receptacle or located at a circumferential or tangential offset with regards to the circumference of the receptacle a net magnetic force applicable to the magnetic coupling member, hence the magnetic effect present to the magnetic coupling member can be increased. Moreover, by having a first and a third electromagnet and by increasing the magnetic field effective on or at the magnetic coupling member the individual magnetic fields that have to be provided by each individual first and third electromagnet can be reduced, thus allowing to further miniaturize the respective electromagnets.

According to a further example the third electromagnet is longitudinally flush with the first electromagnet. Hence, the first electromagnet and the third electromagnet are located at a common longitudinal position with regards to the receptacle. In this way, the first and the third electromagnet may provide somewhat symmetric or identical magnetic fields for driving the magnetic coupling member.

According to a further example the third electromagnet is longitudinally offset from the first electromagnet. Hence, the first electromagnet and the third electromagnet may be longitudinally staggered. The longitudinal offset between the first and the second electromagnet may be defined by the geometry of the core. At as an example, the first leg of the core of the third electromagnet may the located or arranged at a longitudinal position between the first and second legs of the core of the first electromagnet; or vice versa. In this way, the longitudinally extending gap between the first leg and the second leg of e.g. the first electromagnet can be effectively bridged by a leg of the core of the third electromagnet.

According to a further example the third electromagnet is longitudinally shifted relative to the first electromagnet by about half of a longitudinal extent of one of the first electromagnet and the third electromagnet. Typically, the longitudinal extent of the first electromagnet is identical to the longitudinal extent of the third electromagnet. Then, and by longitudinally shifting the position of the third electromagnet by half of the longitudinal extent of the third or first electromagnet there can be provided a longitudinally shifted or staggered arrangement of alternating magnetic poles along the circumference of the receptacle.

With such a longitudinal offset arrangement magnetic forces generated by electromagnets arranged tangentially and/or circumferentially offset there can be provided a rather homogeneous mechanical force on the magnetic coupling member as the magnetic coupling member is moved, e.g. along the longitudinal direction.

According to a further example the drive mechanism comprises a fourth electromagnet longitudinally offset from the third electromagnet. The third electromagnet and the fourth electromagnet form a second longitudinally extending array of electromagnets.

Typically, and with any array or row of electromagnets as described herein, the individual magnets of a row or array of electromagnets of an array of electromagnets is sequentially provided with an electric current, such that a resulting magnetic field generated by the individual electromagnet of an array of electromagnets travels and/or advances along the longitudinal direction.

By way of a fourth electromagnet there can be provided a first and a second array of numerous electromagnets. Also here, the second longitudinally extending array of electromagnets is not limited to only two electromagnets. It may comprise even further electromagnets, such as described above with the first array of electromagnets. The total number of electromagnets of each array of electromagnets may be 3, 4, 5, 6 or even more than 8 electromagnets. The specific number of electromagnets of an array or row of electromagnets may be governed by the size of the individual electromagnets and by the elongation of the receptacle or medicament container.

Typically, the second longitudinally extending array of electromagnets may be substantially identical to the first array of electromagnets. When the first electromagnet and the third electromagnet are arranged at a longitudinal offset as described above this may equally apply to the respective array of electromagnets. Hence, the first array of electromagnets may be located longitudinally offset from the second array of electromagnets. Hence, the longitudinal shift or longitudinal offset of the second longitudinally extending array of electromagnets relative to the first longitudinally extending array of electromagnets may be in a range of half a longitudinal extent of one of the electromagnets.

Typically, the first array of electromagnets extends parallel to the second array of electromagnets. The first array of electromagnets and the second array of electromagnets are typically located at a circumferential or tangential offset with regards to the circumference of the receptacle for the medicament container.

This way and by having a first and a second longitudinally extending array of electromagnets a magnetic coupling between the individual electromagnets and the magnetic coupling member and hence a respective longitudinal force effect onto the magnetic coupling member can be homogenized.

According to a further example the first array of electromagnets and the second array of electromagnets are arranged on radially opposite sides of the receptacle. With other words, the first array of electromagnets may be located diametrically opposite to the second array of electromagnet as seen in cross-section through the receptacle. In this way magnetic forces provided by the individual electromagnets of the first and the second array of electromagnets can be symmetrically applied to the magnetic coupling member, thus allowing to homogenize a force effect onto the magnetic coupling member.

Of course, and with first and second arrays of electromagnets those electromagnets being arranged at a common or at an overlapping longitudinal position are provided with a driving current rather simultaneously. With a longitudinal offset between the electromagnets of the first array of electromagnets and electromagnets of the second array of electromagnets there may be a respective offset for applying a driving current to the individual electromagnets.

The driving current may be provided as a constant driving current, thus representing a rectangular function over time. With other examples the driving current may comprise or describe a ramp-function, e.g. smoothly increasing and/or decreasing over time. With other examples there may be provided a driving current with a sinusoidal amplitude or strength over time.

According to a further example the drive mechanism comprises a third longitudinal array of electromagnets. The first array of electromagnets, the second array of electromagnets and the third array of electromagnets are then equidistantly spaced along the circumference of the receptacle. The first array, the second array and the third array all extend substantially parallel to each other. They may also extend parallel to the longitudinal direction as defined by the receptacle or by the medicament container. An equidistant spacing between the individual arrays is of particular benefit to provide a homogeneous magnetic coupling to the magnetic coupling member, which is in mechanical engagement with the stopper. For example and with three arrays of electromagnets, the angular distance between neighboring arrays of electromagnets may be about 120°.

With further examples and when the drive mechanism comprises e.g. four arrays of electromagnets the angular distance between neighboring arrays or rows of electromagnets is about 90°.

In another aspect the present disclosure relates to a drug delivery device for dispensing a liquid medicament. The drug delivery device comprises a housing configured to hold and/or to receive a cartridge. Typically, the cartridge is filled with a liquid medicament and is sealed by a stopper. Typically, the stopper seals a proximal longitudinal end of the cartridge and is movable relative to the cartridge or relative to a tubular-shaped barrel of the cartridge. The drug delivery device further comprises a drive mechanism as described above. The drive mechanism may be integrated into the drug delivery device. It may be arranged and fixed inside the housing of the drug delivery device. The receptacle of the drive mechanism, which may be e.g. enclosed by one or numerous electromagnets of the drive mechanism, may be integrated into the housing of the drug delivery device. Hence, the receptacle of the drive mechanism may be equivalent or identical to a receptacle of the drug delivery device sized and configured to accommodate the medicament container, which may be implemented as a cartridge.

When a medicament container, e.g. a cartridge, is appropriately assembled or arranged inside the housing a driving current can be appropriately applied to the at least first electromagnet, thereby generating a magnetic field magnetically interacting with the magnetic coupling member, which is mechanically engaged with the stopper of the medicament container.

According to a further example the drug delivery device further comprises a cartridge which is filled with a liquid medicament and which is arranged inside or fastened to the housing of the drug delivery device.

With some examples the drug delivery device comprises an injection device, such as a hand-held injection device. With other examples the drug delivery device comprises an infusion device, e.g. an infusion pump. With other examples the drug delivery device may comprise an inhaler.

Generally and with the drive mechanism as described above the magnetic coupling between the at least one or numerous electromagnets arranged along and/or enclosing the longitudinal receptacle there can be provided a rather constant and/or homogeneous force onto the stopper that can be maintained over a comparatively large distance as the stopper is moved along the elongation of the medicament container. Moreover, the longitudinal displacement and/or movement of the stopper can be precisely controlled by appropriately adjusting at least one of a strength, a direction and a frequency of a driving current on each one of the electromagnets contributing to an array of electromagnets.

This way, a rather strong as well as a variable and electronically adjustable force can be applied onto the stopper.

The present proposed implementation with at least a first and/or numerous electromagnets allows to realize a comparatively short overall longitudinal extension of the drive mechanism. Hence, an elongated piston rod extending in proximal extension of the longitudinally extending cartridge is no longer required. In effect, the longitudinal size of the housing of the drive mechanism or of the housing of the drug delivery device might be predominately defined by the longitudinal extent of the cartridge.

Moreover, the implementation of a at least a first electromagnet for moving a stopper of a cartridge is of particular use for reusable drug delivery devices. Here and especially when the magnetic coupling member is implemented as a permanent magnet there can be provided repulsive and attractive forces in longitudinal direction between an array of electromagnets and the magnetic coupling member. This way, a bidirectional longitudinal force transfer can be enabled between an array of electromagnets and the magnetic coupling member, e.g. in proximal direction as well as in distal direction from the outer coupling member towards the inner coupling member.

In addition, there can be provided a maximum force in both opposite longitudinal directions. Further, and in addition and compared to preloaded force generating solutions the magnetic coupling can be transferred into a rather force-free initial configuration or storage configuration, in which there is effectively no magnetic interaction between the electromagnets and the magnetic coupling member. This can be easily achieved by deactivating a driving current of the electromagnets.

The magnets as described herein may be implemented as permanent magnets. They may comprise or may be composed of, for example, at least one of the following materials comprising sintered Neodymium-Iron-Boron (NdFeB), preferably with a medical-grade coating, Samarium-Cobalt (SmCo) and Aluminium-Nickel-Cobalt (AlNiCo).

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 shows an example of a drug delivery device provided with a drive mechanism according to a first example,

FIG. 2 shows the drive mechanism of FIG. 1 without the housing,

FIG. 3 shows a cross-section through the drive mechanism in a first configuration,

FIG. 4 shows a cross-section of a further example of the drive mechanism,

FIG. 5 shows a further example of a drive mechanism,

FIG. 6 shows another example of a drive mechanism,

FIG. 7 shows a further example of a drive mechanism in a first configuration,

FIG. 8 shows another configuration of the drive mechanism of FIG. 7,

FIG. 9 is illustrative of numerous diagrams showing a magnetic force applied to the magnetic coupling member versus its displacement along the medicament container,

FIG. 10 shows a first configuration of the drive mechanism,

FIG. 11 shows a second configuration of the drive mechanism and

FIG. 12 shows a third configuration of the drive mechanism,

FIG. 13 shows a drive mechanism comprising a first and a second array of electromagnets,

FIG. 14 shows an example with 3 a of electromagnets, and

FIG. 15 shows an example of a drive mechanism comprising four individual arrays of numerous electromagnets.

### DETAILED DESCRIPTION

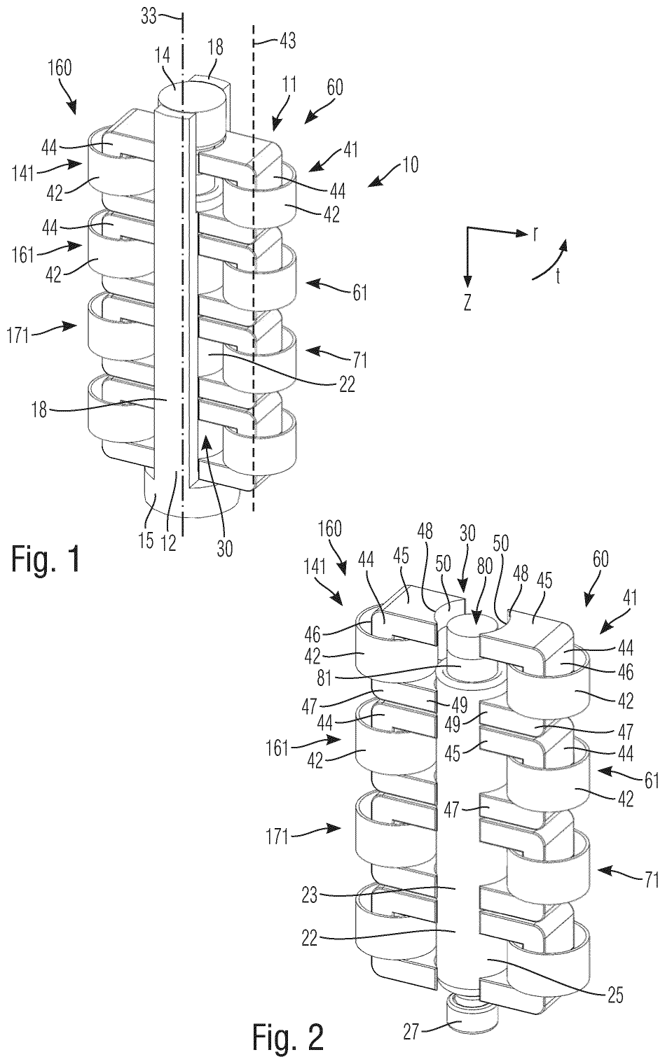

The drug delivery device 10 as illustrated in FIGS. 1-4 comprises a drive mechanism 11 and a housing 12. The drive mechanism 11 is integrated into or arranged inside the housing 12. The housing 12 may comprise a receptacle 30 sized and configured to receive and/or to hold, i.e. to fix a medicament container 22, which in the present case is implemented as a tubular-shaped cartridge. The cartridge may comprise a vitreous body. The cartridge comprises a tubular-shaped barrel 23, e.g. of vitreous material. The cartridge or container 22 is sealed in proximal longitudinal direction by a stopper 24. The stopper 24, typically made of an elastomeric material is movable in longitudinal direction inside the tubular-shaped barrel 23. The interior volume of the cartridge 22 is confined by the barrel 23, the stopper 24 and an outlet 27, which is provided at the distal end of the cartridge 22. The cartridge is fillable or is filled with a liquid medicament 21. The outlet 27 of the cartridge 22 is typically covered or closed by a seal 28. The seal 28 may comprise a rubber disc pierceable by an injection needle 29 in order to expel a dose of the liquid medicament 21 from the interior of the cartridge 22 by displacing or urging the stopper 24 towards the outlet 27.

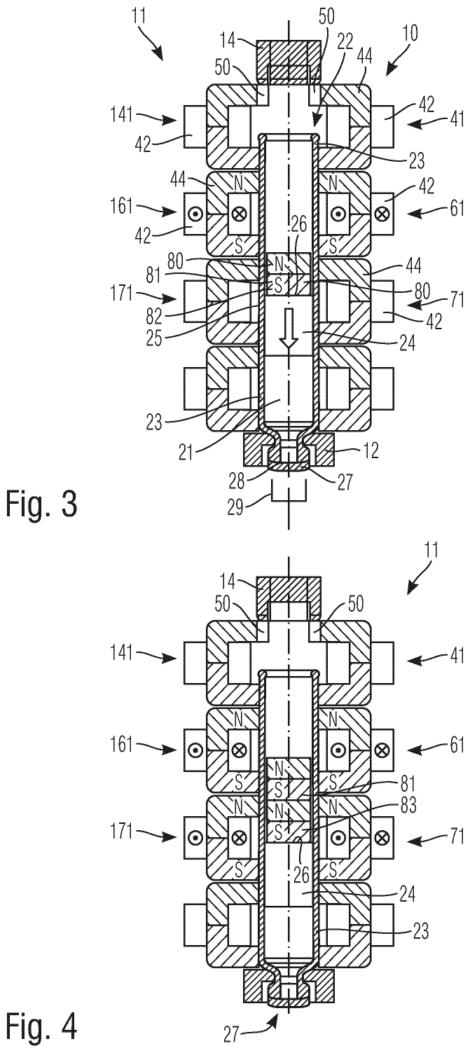

The outlet 27 of the cartridge 22 may be connected to an injection needle 29, which is only schematically illustrated in FIG. 3. With other examples the outlet 27 may comprise a standardized connector, such as a Luer-type connector way of which an infusion line could be connected with the interior of the cartridge 22 in a fluid transferring manner.

With the presently illustrated example the cartridge 22 is fixed inside the housing 12. It may be detachably fixed inside the housing. Hence, the housing may comprise some kind of a detachable fastener or movable closure by way of which there can be provided access to the interior of the compartment 14 so as to replace the cartridge 22 when empty.

The drive mechanism 11 comprises at least a first electromagnet 41. The first electromagnet 41 comprises an electromagnetic coil 42 and a core 44. The core 44 extends through the electromagnetic coil 42. A radial center 43 of the coil 42 is located outside the receptacle 30, which receptacle 30 is sized and shaped to accommodate and/or to receive the medicament container 22. In the illustration of FIG. 1 the housing 12 comprises a base portion 15 forming a mount for the distal end of the medicament container 22. The base portion 15 may be configured to fasten the medicament container 22. As illustrated in the cross-section of FIGS. 3-8 the base portion 15 of the housing 12 may be sized and configured to mechanically engage with a neck of the medicament container 22. Hence the medicament container 22 comprises a shoulder at a distal end of a tubular portion. The shoulder merges into a radially narrowed neck and the neck further extends into a radially widened head. Insofar the neck forms or constitutes a circumferential groove configured to engage with a complementary shaped radial protrusion, e.g. provided at or in the housing 12.

The housing 12 may further comprise a closure 14 by way of which the proximal end of the receptacle 30 can be closed. The housing 12 may further comprise a sidewall 18. The sidewall may radially confine the receptacle 30. The closure may be connectable to the base portion 15 via the sidewall 18. As illustrated in FIG. 1 the sidewall 18 comprises at least an elongated rod or slat-like profile extending in longitudinal direction (z) and extending along the elongation of the receptacle 30.

In the illustration of FIG. 2 the housing 12 is not illustrated. FIG. 2 provides an unobstructed view of numerous electromagnets 41, 61, 71, 141, 161 and 171. The electromagnets 41, 61, 71, 141, 161, 171 may be attached and/or fastened to the housing 12. They may be connected or attached to any of the opposing side walls 18. As illustrated in FIG. 1, the numerous electromagnets 41, 61, 71, 141, 161, 171 are substantially identical. Each electromagnet comprises an electromagnetic coil 42 with a core 44. The core 44 comprises a U-shaped structure. The core 44 comprises a first leg 45, a second leg 47 and a base section 46.

The first leg 45 and the second leg 47 are interconnected by the base section 46. The base section 46 extends through the center of the coil 42. It may slightly protrude from the opposite longitudinal ends of the coil 42. The legs 45, 47 extend at an angle of about 90° from the elongation of the base section 46. In the illustrated example, the first leg 45 and the second leg 47 extend in radial direction. The legs 45, 47 extend in radial direction (r), with regard to the e.g. tubular shape of the receptacle 30.

Both legs 45, 47 extend substantially parallel. They extend from the same radial side of the base section 46. The legs 45, 47 extend towards the receptacle 30. Each leg 45, 47 comprises a free and 48, 49 facing away the base section 46. Each leg 45, 47 further comprises an end face 50 at the free end 48, 49. As illustrated in FIG. 2 the end faces 50 of the legs 45, 47 are of concave shape. They are sized and shaped to accommodate the barrel 23 of the medicament container 22 or cartridge. The tangential extent, hence the extension of the end faces 50 in tangential direction is larger than 30°, larger than 45° or larger than 60° of the outer circumference of the receptacle 30 or barrel 23.

With some examples and as for instance illustrated in FIG. 2 the end faces 50 of the free ends 48, 49 of the legs 45, 47 of the core 44 form a sidewall or sidewall structure or contribute to the a sidewall or sidewall structure of the receptacle 30. Hence, the barrel 23 of the medicament container 22 may be in mechanical engagement or mechanical abutment with the free ends 48, 49 of the core 44.

The free ends 48, 49 of the core 44 form opposite poles of the electromagnet 41. The form or constitute an iron circuit of the electromagnet 41. When an electric current is applied to the electromagnetic coil 42 the magnetic field induced by the electric current is magnified by the core 44 and magnetic poles of opposite type, hence a magnetic north pole and a magnetic south pole are provided at the free ends 48, 49 of the legs 45, 47. By changing the direction of the current in the electromagnetic coil 42 the direction of the magnetic field can be inverted.

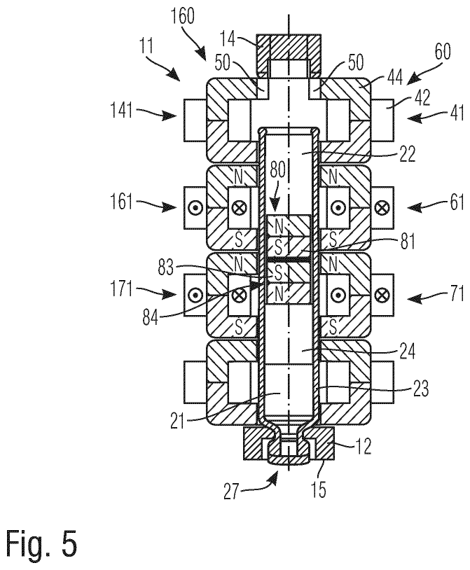

The drive mechanism 11 further comprises a magnetic coupling member 80. The magnetic coupling member 80 comprises a magnet 81, implemented as a permanent magnet. With the illustrated examples the magnet 81 of the magnetic coupling member 80 comprises a rod magnet 82. Alternatively, it may comprise a ring magnets. With the example of FIGS. 4 and 5 the magnetic coupling member 80 even comprises 2 magnets 81, 83 arranged longitudinally adjacently.

The magnetic coupling member 80 is in direct or indirect mechanical engagement with the stopper 24 of the medicament container 22. In the illustrated examples the magnetic coupling member 80 is in longitudinal abutment with a proximally facing end face 26 abutment face of the stopper 24. With other examples (not illustrated) the magnetic coupling member 80 could be integrated into the stopper 24. It might be embedded into the stopper 24. The magnetic coupling member 80 may be fixed to the stopper 24. This way the magnetic coupling member 80 can provide a distally directed and/or a proximally directed force onto the stopper 24 in order to induce a distally and/or a proximally directed movement of the stopper 24 relative to the barrel 23.

In principle, the drive mechanism 11 only requires a single electromagnet 41. With the core 44 comprising a U-shaped structure and comprising first and second legs 45, 47 separated in longitudinal direction (z) there can be established a respective magnetic field extending in longitudinal direction (z), hence along or parallel to the elongation of the receptacle 30. By applying an electric current to the coil 42 a respective magnetic field builds up between the free ends 48, 49 of the core 44 interacting magnetically with the magnetic coupling member 80 and thereby urging the magnetic coupling member 80 in longitudinal direction (z) relative to the barrel 23.

With the presently illustrated examples there are provided numerous electromagnets 41, 61, 71, 141, 161, 171. As illustrated for instance in FIGS. 1 and 2 there is provided a first electromagnet 41 and a second electromagnet 61. The first and the second electromagnets 41, 61 are arranged longitudinally adjacently. Here, the second electromagnet 61 is located distally from the first electromagnet 41. The first electromagnet 41 and the second electromagnet 61 form or constitute a first array 60 of electromagnets 41, 61. As illustrated the first array 60 comprises four electromagnets 41, 61, 71 and one further electromagnet.

The electromagnets 41, 61, 71 are arranged in a longitudinal non-twisting, rather straight row. The first array 60 of electromagnets extends parallel to the longitudinal direction (z) and hence parallel to the elongation of the receptacle 30. The further electromagnets 61, 71 are substantially identical to the first electromagnet 41. There is further provided a second array 160 of electromagnets. The second array 160 comprises a third electromagnet 141 and a fourth electromagnet 161. Moreover, the second array of electromagnets 160 also comprises four electromagnets 141, 161, 171 altogether.

The first electromagnet 41 of the first array 60 of electromagnets and the third electromagnet 141 of the second array 160 are arranged at the same longitudinal position. They are provided on opposite radial sides of the receptacle 30. Here, the free ends 48, 49 of the core 44 of the first electromagnet 41 are diametrically opposite to the free ends 48, 49 of the core 44 of the third electromagnet 141.

When applying identical currents on the first and the third electromagnet 41, 141 at a time there will be provided a twofold and hence a rather radially symmetric magnetic field in the region between the first and third electromagnet 41, 141, thus improving a magnetic interaction with the magnetic coupling member 80. In this way, a rather homogeneous and strong magnetic force can be applied onto the magnetic coupling member 80.

As the magnetic coupling member 80 is subject to a longitudinal displacement, e.g. in distal direction, i.e. towards the outlet 27, the second and the fourth electromagnet 61, 161 may take over a respective generation of a magnetic field operable to apply a respective distally directed force onto the magnetic coupling member 80 and hence onto the stopper 24. This situation is e.g. illustrated in FIG. 5.

As the magnetic coupling member 80 and hence the stopper 24 are moved further in distal direction the function of the second and fourth electromagnets 61, 161 of the first and second arrays 60, 160 of electromagnets may be substituted by the further electromagnets 71, 171 of the respective arrays 60, 160, which are located longitudinally adjacently. Accordingly, and for applying a rather constant and sufficient driving force onto the stopper 24, electric currents applied to individual electromagnets 41, 61, 71, 141, 161, 171 may be individually controlled by a controller (not illustrated). The magnitude of an electric current applied to the individual electromagnets 41, 61, 71, 141, 161, 171 can be adjusted in accordance to an intended force effect to be applied onto the magnetic coupling member 80. Also, and by changing the direction of an electric current in the respective electromagnetic coils 42 the resulting magnetic force as provided by numerous electromagnets 41, 61, 71, 141, 161, 171 can be inverted accordingly.

Typically and for providing a rather smooth displacement of the magnetic coupling member 80 it is intended that electromagnets 41, 141 of different arrays 60, 160, which are located at the same longitudinal position, are synchronously activated by an electric current. With the example of FIG. 4 the magnetic coupling member 80 comprises a first magnet 81 and a second magnet 83. Here, the two magnets 81, 83 are both implemented as rod magnets 82. They are arranged in an attractive configuration. Hence, the magnetic south pole of the magnet 81 faces the magnetic north pole of the magnet 83. Hence, the magnets 81, 83 attract each other.

With two rod magnets 81, 83 arranged in a longitudinal row, the overall longitudinal extent of the magnetic coupling member 80 can be enlarged compared to the configuration of FIG. 3. This way, the magnetic interaction between the individual electromagnets 41, 61, 71, 141, 161, 171 and the magnetic coupling member 80 can be also modified by varying the configuration, e.g. the mutual assembly or orientation of the rod magnets 81, 83 of the magnetic coupling member 80.

With the example of FIG. 5, the magnetic coupling member 80 also comprises two rod magnets 81, 83. But here and contrary to the configuration of FIG. 4 the magnets 81, 83 are oriented in a repelling configuration. There, the south pole of the magnet 81 faces the south pole of the magnet 83. Here, the magnetic coupling member 80 may comprise a mount 84 or a base for mechanically interconnecting the two repelling magnets 81, 83.

The two magnets 81, 83 produce a rather strong repelling magnetic field, by way of which the magnetic coupling of the magnetic coupling member 18 with the electromagnets 41, 61, 71, 141, 161, 171 can be increased. Here, the strength of the magnetic coupling can be increased compared to a configuration as illustrated in FIG. 4.

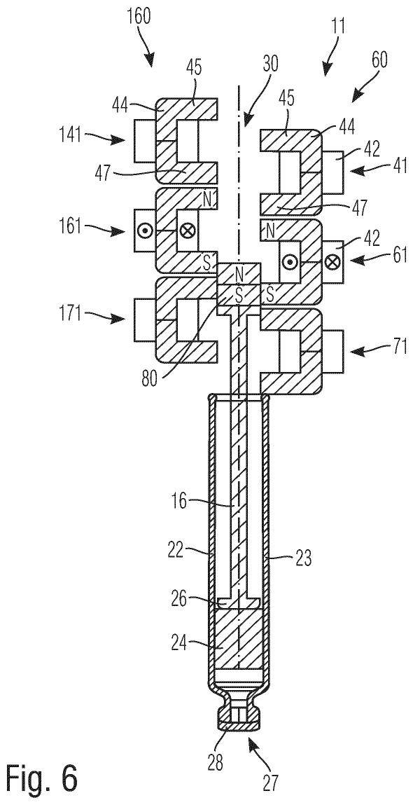

With the further example of FIG. 6 the magnetic coupling member 80 is located at a longitudinal distance from the stopper 24. There is provided a rod that is in abutment with the proximal end face 26 of the stopper 24. An opposite end of the rod 16 supports the magnetic coupling member 80. A force effect acting on the magnetic coupling member 80 is transferred to the stopper 24 by the rod 16. Moreover and as illustrated in FIG. 6 the drive mechanism 11 comprises a first array 60 and a second array 160 of individual electromagnets 41, 61, 71, 141, 161, 171.

In contrast to the example of FIGS. 3-5 the arrays 60, 160 are longitudinally offset with respect to each other. Hence, the electromagnets 41, 61, 71 of the first array 60 are longitudinally offset from the electromagnets 141, 161, 171 of the second array 160. As illustrated the second leg 47 of the core 44 of the third electromagnet 141 is located longitudinally between the first leg 45 and the second leg 47 of the core 44 of the first electromagnet 41. Of course and as described before, the legs 45, 47 of the cores 44 of the electromagnets 41, 141 are located on opposite radial sides of the receptacle 30.

In this way there can be provided a rather staggered arrangement of an alternating sequence of electromagnets on either side of the receptacle 30. This allows to further spatially homogenize the magnetic coupling between first and second arrays 60, 160 of electromagnets with the magnetic coupling member 80.

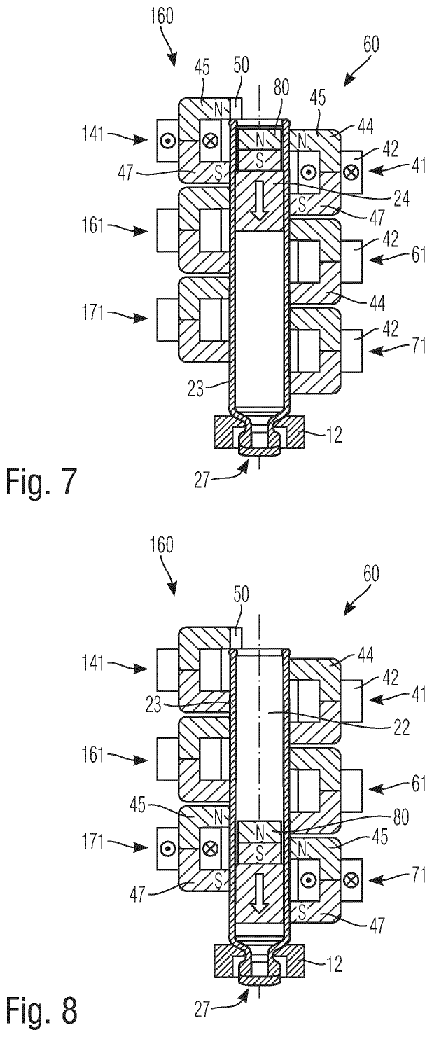

In the further examples of FIGS. 7 and 8 the first and second arrays 60, 160 of electromagnets 41, 61, 71, 141, 161, 171 are longitudinally offset by half of a longitudinal extent of an individual electromagnet 41 and/or by half of the longitudinal extent of a core 44.

As illustrated in FIG. 7 and when the stopper 24 is close to a proximal end of the barrel 23 the first electromagnet 41 of the first array 60 and the first electromagnet 141 of the second array 160 are active. As the magnetic coupling member 80 and hence as the stopper 24 is moved in distal direction, i.e. towards the outlet 27, the longitudinally adjacent electromagnets 61, 161 will be activated at the expense of the first electromagnet 41 and the third electromagnet 141. Here, the third electromagnet 141, being the most proximally arranged electromagnet may be switched off at the benefit of the fourth electromagnet 161, which will be activated.

Thereafter and as the magnetic coupling member 80 further moves in distal direction the first electromagnet 41 may be deactivated while the third electromagnet 61 will be activated. In FIG. 8, an end configuration is illustrated, where the further electromagnets 71, 171 of the first and second array 60, 160 are finally activated until the stopper 24 reaches a distal end position.

In the example of FIGS. 10-12 a sequence of use of an example of a first electromagnet 41 and a third electromagnet 141 located at a longitudinal offset on opposite sides of the barrel 23 is illustrated. There, and in an initial configuration as illustrated in FIG. 10 the electromagnets 41, 141 are both activated by a driving current flowing in the same direction. Hence, the north pole of the electromagnet 41 and the north pole of the electromagnet 141 face towards the proximal end. The south poles are provided at the distal end.

Now and as the magnetic coupling member 80 has been moved in distal direction as illustrated in FIG. 11 the direction of the current of the third electromagnet 141 may be inverted, thus inverting the magnetic field of the third electromagnet 141. The change in magnetization of the third electromagnet 141 further induces a driving force onto the magnetic coupling member 80 towards the distal direction. When a configuration as illustrated in FIG. 12 has been reached the polarity and hence the magnetic field of the first electromagnet 41 may be also inverted.

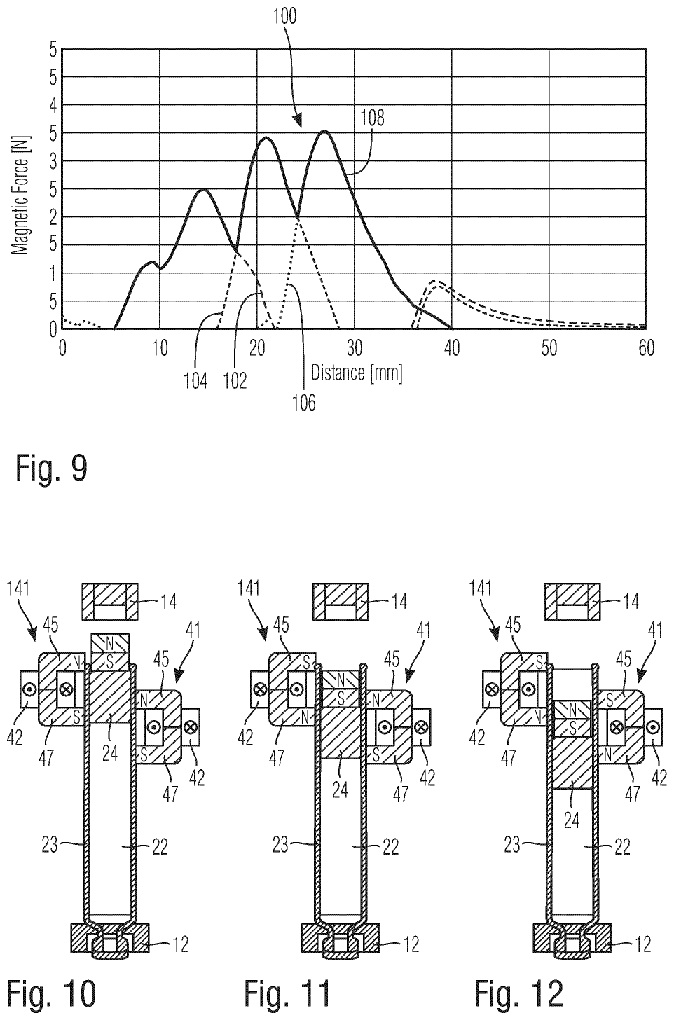

The resulting magnetic forces that are applied on the magnetic coupling member 80 are further illustrated in the diagram 100 of FIG. 9. Here, the graph 102 illustrates the force applied on the magnetic coupling member 80 when the first and third electromagnets 41, 141 are in the configuration of FIG. 10. The graph 104 shows the magnetic force over a longitudinal displacement of the magnetic coupling member 80 when the first and third electromagnets 41, 141 are in the configuration according to FIG. 11 and the graph 106 illustrates the magnetic force applied onto the magnetic coupling member 80 when the first and third electromagnets 41, 141 are in the configuration of FIG. 12. The graph 108 represents the resulting forces as obtained by alternatively switching the magnetizations of the third and first electromagnets 141, 41 as the magnetic coupling member 80 is subject to a successive longitudinal displacement towards the outlet 27.

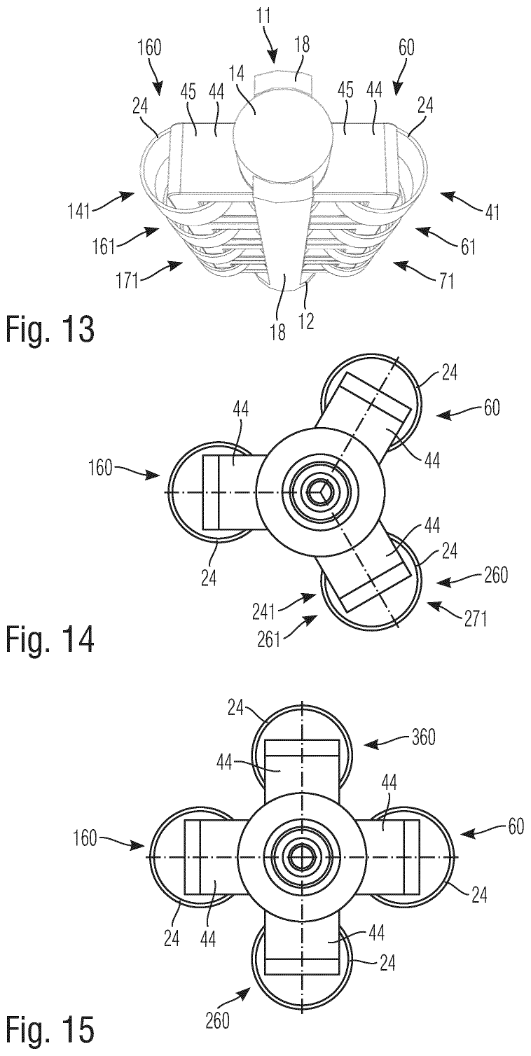

In FIG. 13 the drive mechanism 11 as illustrated in FIG. 1 is shown in a perspective view from the top. Here, the closure 14 as well as the tangential extent of the core 44 are particularly illustrated.

With the example of FIG. 14 there are provided three arrays 60, 160, 260 of numerous electromagnets. Here, the third array 260 of electromagnets 241, 261, 271 may be substantially identical to any of the first array 60 and second array 160 of electromagnets. The individual electromagnets 241, 261, 271 are only indicated in FIG. 14. The single and individual electromagnets 241, 261 271 are arranged in a longitudinal row and are not particularly visible due to the perspective of the illustration.

With the example of FIG. 14 the arrays 60, 160, 260 of electromagnets are arranged equidistantly with regard to the circumferential or tangential direction (t). Hence, adjacently located arrays of electromagnets 60, 160, 260 are separated by about 120°.

With the further example of FIG. 15 there are provided altogether four rows or arrays 60, 160, 260, 360 of electromagnets. There, the arrays each extend in longitudinal direction (z) and comprise numerous electromagnets 41, 61, 71. The arrays 60, 160, 260, 360 are separated equidistantly with regards to the circumferential or tangential direction. Here, the angular distance between neighboring or adjacently located arrays 60, 160, 260, 360 of electromagnets is about 90°.

With the examples of three or four electromagnets 60, 160, 260, 360 the electromagnets 41, 61, 71 of e.g. the first array 60 may be arranged at the same longitudinal position as the electromagnets 141, 161, 171 of e.g. the second array 160.

With some examples the electromagnets 41, 61, 71 of e.g. the first array 160 may be arranged at a longitudinal offset from the electromagnets 141, 161, 171, 241, 261, 271 of any of the further arrays 160, 260, 360. Here, the longitudinal offset may be a ⅓ or ¼ of the longitudinal extent of an individual electromagnet 41. This way, and for applying a rather constant longitudinal force effect onto the magnetic coupling member 80 the individual electromagnets 41, 141, 241 of the arrays 60, 160, 260 of electromagnets may be sequentially activated following their helical order of their spatial arrangement.

### REFERENCE NUMBERS

10 drug delivery device
11 drive mechanism
12 housing
14 closure
15 base portion
16 rod
18 side wall
21 medicament
22 cartridge
23 barrel
24 stopper
25 outside surface
26 proximal face
27 outlet
28 seal
29 injection needle
30 receptacle
33 radial center
41 electromagnet
42 electromagnetic coil
43 radial center
44 core
45 leg
46 base section
47 leg
48 free end
49 free end
50 end face
60 array
61 electromagnet
71 electromagnet
80 magnetic coupling member
81 magnet
82 rod magnet
83 magnet
84 mount
141 electromagnet
160 array
161 electromagnet
171 electromagnet
241 electromagnet
260 array
261 electromagnet
271 electromagnet
360 array

1. A drive mechanism (11) for moving a stopper (24) of a medicament container (22), wherein the stopper (24) is movably disposed inside a longitudinally extending barrel (23) of the medicament container (22) and is movable in a longitudinal direction (z) relative to the barrel (23), the drive mechanism (11) comprising:
a receptacle (30) sized to receive the medicament container (22),
a first electromagnet (41) comprising an electromagnetic coil (42) and a core (44) extending through the electromagnetic coil (42), wherein a radial center (43) of the electromagnetic coil (42) is located outside the receptacle (30),
a magnetic coupling member (80) mechanically engagable with the stopper (24) and configured for insertion into the barrel (23),
wherein the first electromagnet (41) is operable to generate a magnetic field causing the magnetic coupling member (80) to move in the longitudinal direction (z).

2. The drive mechanism (11) according to claim 1, wherein the radial center (43) of the electromagnetic coil (42) is located at a radial offset from a radial center (33) of the receptacle (30).

3. The drive mechanism (11) according to any one of the preceding claims, wherein the core (44) comprises a first leg (45), a base section (46) and a second leg (47), wherein the base section (46) extends through the electromagnetic coil (42) and wherein the first leg (45) and the second leg (47) extend in radial direction (r) towards the receptacle (30).

4. The drive mechanism (11) according to claim 3, wherein the first leg (45) and the second leg (47) are separated in longitudinal direction (z) and are interconnected via the base section (46).

5. The drive mechanism (11) according to claim 3 or 4, wherein the core (44) is located radially outside the receptacle (30), and wherein a free end (48) of the first leg (45) and a free end (49) of the second leg (47) is facing radially inwardly towards the receptacle (30).

6. The drive mechanism (11) according to claim 5, wherein at least one of the free ends (48, 49) of the first leg (45) and the second leg (47) comprises a radially inwardly facing end face (50) with a concave shape as seen in a tangential direction (t).

7. The drive mechanism (11) according to any one of the preceding claims, further comprising a second electromagnet (61) longitudinally offset from the first electromagnet (41), wherein the first electromagnet (41) and the second electromagnet (61) form a first longitudinally extending array (60) of electromagnets (41, 61, 71).

8. The drive mechanism (11) according to any one of the preceding claims, further comprising a third electromagnet (141) tangentially offset from the first electromagnet (41).

9. The drive mechanism (11) according to claim 8, wherein the third electromagnet (141) is longitudinally flush with the first electromagnet (41).

10. The drive mechanism (11) according to claim 8, wherein the third electromagnet (141) is longitudinally offset from the first electromagnet (41).

11. The drive mechanism (11) according to claim 10, wherein the third electromagnet (141) is longitudinally shifted relative to the first electromagnet (41) by about half of a longitudinal extent of one of the first electromagnet (41) and the third electromagnet (141).

12. The drive mechanism according to any one of the preceding claims 8-11, further comprising a fourth electromagnet (161) longitudinal offset from the third electromagnet (141), wherein the third electromagnet (141) and the fourth electromagnet (161) form a second longitudinally extending array (160) of electromagnets (141, 161, 171).

13. The drive mechanism according to claim 12, wherein the first array (60) of electromagnets (41, 61, 71) and the second array (160) of electromagnets (141, 161, 171) are arranged on radially opposite sides of the receptacle (30).

14. The drive mechanism according to claim 12, further comprising a third longitudinally extending array (260) of electromagnets (241, 261, 271), wherein the first array (60) of electromagnets (41, 61, 71), the second array (160) of electromagnets (141, 161, 171) and the third array (260) of electromagnets (241, 261, 271) are equidistantly spaced along the circumference of the receptacle (30).

15. A drug delivery device for dispensing a liquid medicament, the drug delivery device comprising:
a housing (12) configured to hold and/or to receive a cartridge (22) filled with the liquid medicament and sealed by a stopper (24), and
a drive mechanism (11) according to any one of the preceding claims.

16. The drug delivery device (10) according to claim 15, further comprising the cartridge (22) arranged inside or fastened to the housing (12).
